(19) United States
(12) Plant Patent Application Publication
(10) Pub. No.: US 2016/0205852 P1
(43) Pub. Date: Jul. 14, 2016

(54) Verbena plant named 'VEAZ0023'

(50) ;

(71) Applicant: Syngenta Participations AG, Basel (CH)

(72) Inventor: Kathryn Verlinden, Andijk (NL)

(73) Assignee: Syngenta Participations AG, Basel (CH)

(21) Appl. No.: 14/756,739

(22) Filed: Oct. 6, 2015

Related U.S. Application Data
(60) Provisional application No. 62/102,703, filed on Jan. 13, 2015.

(51) Int. Cl. A01H 5/00

(52) U.S. Cl. PLT/308

(57) ABSTRACT

A new Verbena plant named ‘VEAZ0023’ particularly distinguished by the its medium sized inflorescences, large, deep-red flowers, medium green, serrated leaves, and a compact plant habit.

### LATIN NAME OF THE GENUS AND SPECIES OF THE PLANT CLAIMED

Verbena hybrida

### VARIETAL DENOMINATION

‘VEAZ0023’

### BACKGROUND OF THE NEW PLANT

The present invention comprises a new Verbena, botanically known as Verbena hybrida, and hereinafter referred to by the variety name ‘VEAZ0023’.

‘VEAZ0023’ is a product of a planned breeding program. The new cultivar has medium sized inflorescences, big flowers that are of deep-red color, medium green, serrated leaves, and a compact plant habit.

VEAZ0023’ originated from a cross-pollination made in July 2010 in a greenhouse in Enkhuizen, The Netherlands.

The female parent was an unpatented proprietary plant designated ‘J0397-3’ having a scarlet flower color and an upright plant habit. The male parent was an unpatented proprietary plant designated ‘K0546-3’ having a deep-rose flower color and a compact, spreading plant habit

The resulting seed was sown in February 2011 in a greenhouse in Enkhuizen, The Netherlands. ‘VEAZ0023’ was selected as one flowering plant within the progeny of the stated cross in May 2011.

The first act of asexual reproduction of ‘VEAZ0023’ was accomplished when vegetative cuttings were propagated from the initial selection in June 2011 in a greenhouse in Enkhuizen, The Netherlands.

### BRIEF SUMMARY OF INVENTION

Horticultural examination of plants grown from cuttings of the plant initiated in the fall of 2011, and continuing thereafter, has demonstrated that the combination of characteristics as herein disclosed for ‘VEAZ0023’ are firmly fixed and are retained through successive generations of asexual reproduction.

‘VEAZ0023’ has not been observed under all possible environmental conditions. The phenotype may vary significantly with variations in environment such as temperature, light intensity, and day length.

A Plant Breeder's Right for this cultivar has been applied with the European Union Plant Breeder's Rights Office (CPVO) in Nov. 7, 2013, under No. 2013/2680; and granted EU39741 on Feb. 23, 2015.

The following traits have been repeatedly observed and are determined to be basic characteristics of the new variety. The combination of these characteristics distinguishes this Verbena as a new and distinct variety.

### BRIEF DESCRIPTION OF THE PHOTOGRAPHS

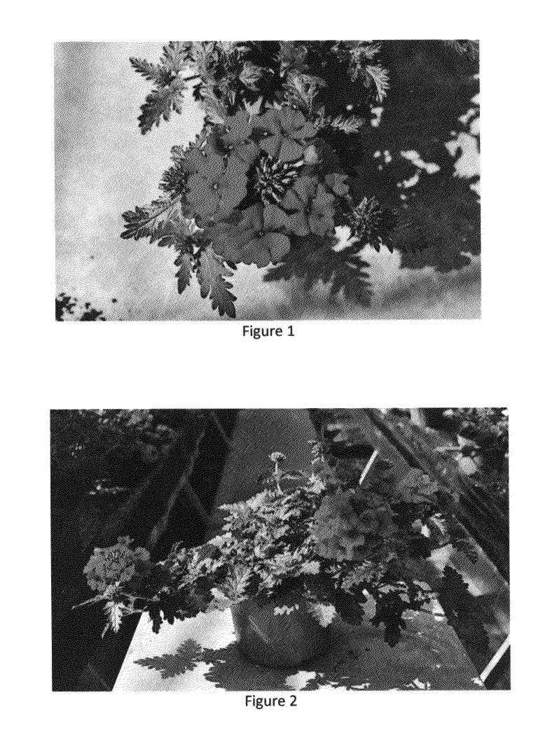

The accompanying photographic drawings show typical flower and foliage characteristics of ‘VEAZ0023’ with colors being as true as possible with an illustration of this type. The photographic drawing in FIG. 1 shows a close view of an inflorescence of the new plant and in FIG. 2 a whole flowering plant.

### DETAILED BOTANICAL DESCRIPTION

The aforementioned photographs were taken in late April 2013 in Andijk, The Netherlands. The plants were about 10 weeks old and were grown in 12 cm pots.

The plant description and measurements were made in Enkhuizen, The Netherlands on May 27, 2015 indoors in natural light. The plants used were about 10 weeks of age and growing in an 11 cm pot in a greenhouse.

Color references are made to The Royal Horticultural Society Colour Chart (R.H.S.) 2001

TABLE 1
DIFFERENCES BETWEEN THE NEW VARIETY ‘VEAZ0023’
AND A MOST SIMILAR VARIETY

| | ‘VEAZ0023’ | ‘Vepita Fire Red’ |
|---|---|---|
| Flower color: | Deep Red | Scarlet Red |
| Flower, size: | Large | Medium |
| Leaf, size: | Bigger | Smaller |
| Plant habit: | Less branching | More branching |

Plant:
   Form, growth and habit.—Herbaceous outwardly spreading, vigorous and free-branching.
   Plant height.—13.0 cm.
   Plant height (inflorescence included).—17.0 cm.
   Plant width.—36.0 cm.
Roots:
   Number of days to initiate and produce roots.—10-12 days at about 22 degrees C.
   Type.—Fine, fibrous, free branching.
   Color.—RHS N155D but whiter.
Foliage:
   Arrangement.—Opposite, simple.
   Immature, leaf color, upper surface.—RHS 137B.
   Immature, leaf color, lower surface.—RHS 137C.
   Mature, leaf color, upper surface.—RHS 137A.
   Mature, leaf color, lower surface.—RHS 136D.
   Length.—About 5 cm.
   Width.—3.0-3.5 cm.
   Shape.—Ovate.
   Base shape.—Cordate.
   Apex shape.—Acute.
   Margin.—Serrate.
   Texture, upper surface.—Hirsute.
   Texture, lower surface.—Hirsute.
   Color of veins, upper surface.—RHS 146A.
   Color of veins, lower surface.—RHS 146B.
   Pattern of veining.—Pinnate.
   Petiole color.—RHS 145B.
   Petiole length.—0.9 cm.
   Petiole width.—0.2 cm.
   Texture.—Hirsute.
Stem:
   Branching characteristics.—Most branches going upright.
   Quantity of main branches per plant.—10-12.
   Color of stem.—RHS 146B.
   Stem length.—15-16 cm.
   Stem width.—0.3 cm.
   Length of internodes.—2-4 cm.
   Texture.—Hirsute.
   Color of peduncle.—RHS 138B.
   Length of peduncle.—2-2.5 cm.
   Peduncle diameter.—0.2 cm.
   Texture.—Canescent.
Inflorescence:
   Type.—Umbel-like, but actually a spike, umbrella-shaped, large.
   Blooming habit.—Flowers continuously through the summer months.
   Lastingness of individual blooms on the plant.—About 4 days in the greenhouse.
   Fragrance.—Slight lavender fragrance.
   Number of inflorescences per plant.—50-62 at various stages.
   Horizontal diameter of inflorescence.—About 5.5 cm.
   Depth of inflorescence.—3.0 cm.
   Number of florets per inflorescence (including any buds at the time).—18-25.
Bud (just when opening/showing color):
   Color.—RHS 62B.
   Length.—0.5 cm.
   Width.—0.4 cm.
   Shape.—Tubular with a bulbous end.
Floret:
   Form.—Sessile; salver-shape, composed of 5 partly fused petals with a base of a narrow tube.
   Immature color, upper surface.—RHS 61C.
   Immature color, lower surface.—RHS 61D.
   Mature color, upper surface.—RHS 46B.
   Mature color, lower surface.—RHS 46C.
   Floret diameter.—2.-2.3 cm.
   Floret length (depth).—2.2 cm.
   Petal length.—1.2 cm.
   Petal width.—1.1 cm.
   Shape.—Obcordate.
   Apex shape.—Emarginate.
   Margin.—Entire.
   Petal texture, upper surface.—Papillose; glabrous.
   Petal texture, lower surface.—Papillose.
   Corolla tube length.—1.4 cm.
   Corolla tube width.—0.2 cm.
   Corolla tube color inside.—RHS 146D.
   Tube color outside.—RHS 145D near corolla to RHS 150C at base.
   Corolla texture, inside.—Papillose, short hair with a few longer hairs.
   Corolla texture, outside.—Glabrous at base, upper part canescent.
   Throat hair color.—RHS 155D but whiter.
Calyx:
   Type.—Five sepals whose margins are fused to each other along their length, with a transparent membrane of less than 0.1 cm in width and with one smaller sepal attached to the base of the calyx.
   Color of sepals.—RHS 143C.
   Length of sepals.—1.1 cm.
   Width of sepals.—0.2 cm.
   Sepal shape.—Linear, but fused.
   Apex shape.—Acute.
   Margins.—Entire.
   Texture.—Densely covered with short hair and many glandular hairs.
Reproductive organs:
   Gynoecium.—
   Pistil.—1.
   Pistil length.—1.6 cm.
   Style color.—RHS N144C.
   Style length.—1.5 cm.
   Stigma color.—RHS 144B.
   Ovary color.—RHS 144B.
   Ovary length.—0.2 cm.
   Ovary width.—0.1 cm.
   Androecium.—
   Stamens.—Anthers and filaments fused to upper half of corolla tube; four anthers with two pollen sacs per anther.
   Color of filaments.—RHS 150C.
   Filament length.—0.2 cm.
   Anther color.—RHS 150C.
   Anther length.—0.15 cm.
   Pollen amount.—None observed.
Fertility/seed set: Neither seed nor fruit production has been observed.
Disease/pest resistance: Resistance to pathogens and pests common to Verbena has not been observed.

What is claimed is:

1. A new and distinct variety of Verbena plant named ‘VEAZ0023’ substantially as illustrated and described herein.
